(19) United States
(12) Patent Application Publication
(10) Pub. No.: US 2017/0156369 A1
(43) Pub. Date: Jun. 8, 2017

(54) Isolation of Plant Oligopeptides and Uses Thereof

(71) Applicant: Rebecca Lynn Scotland, Hamilton, OH (US)

(72) Inventors: Xinqi Liu, Beijing (CN); Rebecca Lynn Scotland, Liberty Twp., OH (US)

(21) Appl. No.: 15/330,415

(22) PCT Filed: Jul. 15, 2015
(86) PCT No.: PCT/IB15/55341

§ 371 Date: Jan. 9, 2017

Related U.S. Application Data
(60) Provisional application No. 62/024,390, filed on Jul. 14, 2014.

(51) Int. Cl. A23J 3/30; C07K 1/34

(52) U.S. Cl. CPC A23J 3/30; C07K 1/34

(57) ABSTRACT

The invention discloses a granular, free-flowing, non-dusting enriched oligopeptide isolate with a narrow, low-molecular-weight distribution derived from legume, seed, grain, marine and other sprouted or un-sprouted plant protein isolates and improved suitability for industrial applications and method for preparing the same. The novel oligopeptide isolate possess fluidity, dispersion, solubility, sensory properties, interaction stability and safety that are consistent and well-suited for applications. The viscosity and clarity of the hydrate are well suited for applications. The product is stable, potent and easily absorbed by the body. The effective method of processing used to produce the oligopeptide isolate includes an ultra-high temperature processing treatment prior to enzymatic hydrolysis, dilution ratio and Brix parameters for hydrolysis and separation, nanofiltration and coupled fluidized bed and spray drying followed by drum drying process. The resulting plant or marine oligopeptide enriched isolate is suitable, not only for nutrient fortification of acidic media, but may be used in a wide variety of conventional applications of protein isolates, including but not limited to, fortification of acidic and non-acidic foods and beverages, emulsification of oils, as a body former in baked goods and foaming agent in products which entrap gases, pharmaceutical, preventative health, dietary supplement, pediatric nutrition, food additive, pet food, animal feed, fertilizer, antioxidant, antimicrobial, cosmetic, surfactant, adhesive and bio-fuel formulations.

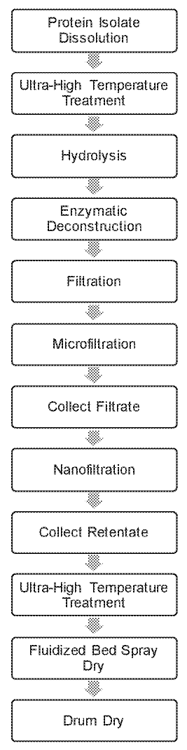

### TECHNICAL FIELD

The present invention generally relates to plant and marine protein oligopeptide enriched isolates and deep processing for producing the same. More particularly, the present invention relates to a high-yield method of isolating a granular, free-flowing, non-dusting, low-molecular-weight oligopeptides with improved suitability for incorporation into industrial applications.

### BACKGROUND ART

The demand for efficient, high-quality protein nutrient sources will be expanded due to the growth of the middle class in emerging countries. Thus, balance of demand and supply is predicted to become tight. Plant and marine sources provide a low ecological burden source of protein nutrients. Furthermore, peptide isolates can serve as a concentrated isolate of stable protein nutrients with a range of applications, as unmodified protein materials tend to be substantially insoluble.

As generally defined, peptide isolates may include substance obtained by acidic, alkaline or enzymatic hydrolysis of protein composed primarily of amino acids, peptides and proteins and may contain impurities consisting chiefly of carbohydrates and lipids along with smaller quantities of miscellaneous organic substances of biological origin. Peptide isolates have documented applications in textiles including applications such as plywood adhesives; aquaculture and agriculture including applications such as promotion of plant rooting, germination, growth and prolong lifetime; biofuels; cosmetic formulation; biopharmaceuticals and absorbent hydrogel formulation; functional food and beverage formulation; enteric diet formulation and dietary supplementation; infant and pediatric nutritional product formulation; animal feed formulation; cell culture growth medium and fermentation processing; baking ingredient to improve resistance against freezing and favorable texture. Peer-reviewed scientific evidence is mounting regarding the role of bioactive peptides as antioxidants, anticoagulants, anti-inflammatory modulators, antibacterial, antifungal and antiviral agents, thermogenic agents, anticancer (colon and prostate), anti-osteoporosis, cell growth and repair modulators, angiotensin-converting enzyme (ACE) inhibitors, in addition to biological signaling mediators involved in a myriad of signaling functions with impact on recovery, lipid metabolism, carbohydrate metabolism, immune function, cardiovascular and bone health, nervous system and brain function, optimizing muscle performance during exercise, digestive satiety and weight management.

Previously, plant peptides have been isolated by: dissolution of the protein isolate; enzymatic hydrolysis followed by enzyme inactivation; separation of peptides from the reaction mixture by solvent extraction, centrifugation, ultrafiltration or chromatography; sterilization; concentration; freeze or spray drying; and deodorization. Enzyme activity has been regulated by adjusting pH, temperature and proteolytic enzyme mixtures (Galvez & de Lumen, 1999). However, goldilocks pH and temperature ranges that optimize enzyme activity tend to maximize protein folding. High steric hindrance results in poorly hydrolyzed proteins, which adversely affects solubility, absorption, potency, sensory properties and interaction stability. Meanwhile, less sterically hindered polypeptides may undergo successive enzymatic hydrolysis resulting in high free amino acid concentrations, which adversely affects absorption, potency, sensory properties, degradation and interaction stability.

The most common technique for separating peptides from the reaction mixture has been centrifugation. The resulting product has a broad molecular weight distribution, skewed toward high-molecular-weight peptides, which adversely affects dispersion, solubility, absorption, potency, sensory properties and interaction stability. The general procedure for the above-described steps is well understood. Conventional high-temperature concentration of peptides produces thermal by-products, which adversely affect solubility, potency and sensory properties. Freeze or spray drying of peptide isolates has resulted in a product with inconsistent particle size and high dust, which adversely affect fluidity, dispersion and solubility resulting in reduced suspension stability and suitability for use in many applications.

The prior art has not disclosed an adequate method for processing plant or marine proteins to produce low-molecular-weight peptide enriched isolate in high yield. Heretofore, such peptide isolates have exhibited broad molecular weight distributions. The resulting solubility, bioavailability, bioactive potency, interaction stability and sensory properties have shown limited biological and chemical suitability for applications. Furthermore, inconsistent particle size, fluidity and dispersion characteristics present physical challenges for industrial applications.

Concerning such, the invention discloses a high-yield method for processing plant and marine protein isolates to produce a uniform, granular, low-molecular-weight oligopeptide enriched isolate with a narrow molecular weight distribution obtained by a novel functional sequence of ultra-high temperature processing treatment prior to enzymatic hydrolysis, dilution ratio and Brix parameters for hydrolysis and separation, nanofiltration, coupled fluidized bed and spray drying followed by drum drying.

### DISCLOSURE OF INVENTION

### Problem to be Solved by the Invention

Processing methods for peptide isolation have been described in the prior art, however, methods enzyme optimization techniques, separation, concentration and drying procedures tend to result in reduced solubility, absorption, potency, desirable sensory properties and interaction stability of the concentrated peptide isolate yielding high free amino acid concentrations, broad molecular weight distribution skewed toward high-molecular-weight peptides, unstable and/or degraded products and inconsistent particle size and high dust, resulting in reduced suitability for use in many applications.

The present invention has accounted for the aforementioned circumstances and embodies enrichment of protein isolates to improve product purity and stability and provide strict control of the product molecular weight range. The object of the present invention is to provide means useful for establishing a superior oligopeptide isolate production system for plant and marine-derived protein isolates.

### Means for Solving Problem

As a result of an extensive study for solving the above problem, the present inventors have derived a method excellent in ability to produce consistent, free-flowing oligopeptides with the desired properties for industrial applications, and have completed the present invention.

The comestible composition may include but is not limited to, a tablet, food, candy, gel, powder, beverages selected from carbonated water, flavored water, carbonated flavored water, spring water, fruit juice, vegetable juice or nectar, coffee, decaffeinated coffee, tea, fruits and products derived from tea, herbal products from tea, decaffeinated tea, wine, champagne, ale, rum, gin, vodka, other liquor, milk obtained from animals, from soybeans, rice, coconut milk or other plant products. Beverage selected from the group consist of, but are not limited to sports beverages, beverage concentrates, hypotonic beverages, soft drinks, strong drinks (shot), sport drinks, hypertonic drinks, energy drinks and isotonic drinks. Nutritional formulations optionally comprise one or more amino acids, antioxidants, fat, vitamins, trace elements, electrolytes, sweeteners, flavors and/or mixtures thereof, caffeine, coloring agents, emulsifying agents, flavor enhancers, food grade acids, minerals, micronutrients, botanical extracts, phytochemicals, preservatives, buffer salts include salts class, stabilizers, thickeners, pharmaceutical ingredients, fiber, prebiotics, probiotics and/or combinations thereof.

Accordingly, the present invention is a concentrated oligopeptide isolate derived from plant or marine protein isolates comprising a higher total proportion of narrowly distributed, low-molecular-weight peptides with a lower total proportion of free amino acids in a free-flowing, uniform granules of 40 to 60 μm particle size with a low moisture content and soluble hydrate at low pH with a method of producing the same comprising ultra-high temperature processing treatment from 130 to 150° C., hydrolysis under conditions of 5 to 20° Bx, separation Brix parameters of 4 to 20° Bx, 1 to 15 ratio water wash after microfiltration, nanofiltration by pulsating flow pressure at 10 to 35° Bx, coupled fluidized bed and spray drying followed by drum drying to form a concentrated granular oligopeptide isolate.

### Effect of the Invention

According to the present invention, it is possible to achieve plant or marine protein oligopeptide enriched isolates in high-yield with fluidity, dispersion, solubility, sensory properties and interaction stability are consistent and well-suited for industrial applications. The hydrate remains clear under acidic and low temperature conditions and the viscosity of the hydrate is low. The product is stable, potent and easily absorbed by the body. The scope of the present invention can be widely used in the form and added as the form to include, but not limited to applications in pharmaceutical, preventative health, dietary supplement, functional food and beverage, pediatric nutrition, food additive, animal feed, fertilizer, antioxidant, antimicrobial, cosmetic, surfactant, adhesive and bio-fuel formulations. The oligopeptide enriched isolate described in the present invention can also be fermented with different types of starter or probiotic cultures or can be combined with all kinds of ingredients such as oils, fats, emulsifiers, carbohydrates, fruit concentrates, flavors, colorants, alcohol, carbon dioxide, thickeners, acidulates, antioxidants, herbs or herb extracts, health promoting compounds like vitamins or bioactive compounds formulate a product which is in line with the marketing needs.

### BRIEF DESCRIPTION OF DRAWINGS

The drawing summarizes the present invention process.

### BEST MODE FOR CARRYING OUT THE INVENTION

Herein a peptide or oligopeptide are defined as a chain of at least two amino acids that are linked through peptide bonds. The terms “peptide” and “oligopeptide” can be used interchangeably as the context requires. A protein consists of one or more chain comprising of more than 30 amino acid residues (polypeptides) linked together by peptide bonds. As used herein a protein hydrolysate, hydrolysate, or hydrolysed protein is the product that is formed by hydrolysis of the protein peptide bonds between amino acids. An enriched hydrolysate being a fraction of the protein hydrolysate, for example enriched in selected peptides or wherein a subset of peptides or polypeptides have been removed from the hydrolysate. So an enriched hydrolysate is preferably a mixture of peptides or a peptide mixture.

The process of the formation of granular isolate concentrate of low-molecular-weight oligopeptides begins with protein isolates. Raw materials may include legume, seed, grain, marine and other sprouted or un-sprouted plant protein isolates. Examples of raw plant and marine proteins include, but are not limited to, protein and polypeptides derived from soybean, pea, corn, canola, Jatropha, palm, peanut, sunflower, coconut, mustard, cotton seed, Palm kernel, olive, safflower, sesame, linseed and microbial proteins or polypeptides from yeast or bacterium. For the purpose of the present invention, whole plant or marine protein isolates may be standard, commoditized plant or marine protein isolates. More specifically, the protein source may be whole or any product or by-product derived from the processing of plant or marine protein sources including but not limited to meal, flakes, grits and flour. The protein source may be used in the full fat form, partially defatted form or fully defatted form. Where the protein source contains an appreciable amount of fat, an oil-removal step is generally required during the process. The protein recovered from the protein source may be the protein naturally occurring in plant or marine sources or the proteinaceous material may be a protein modified by genetic manipulation but possessing characteristic hydrophobic and polar properties of the natural protein.

Protein isolation can be performed by any method known in the art. The general, conventional procedures for protein isolates of various plant or marine origin are described in the prior art. Typically, processing will include isolation of the protein containing portion of the organism, flaking, extraction of fat and decanting insoluble materials, such as fiber and cellulose, followed by pH adjustments. Where the protein isolate starting material contains an appreciable amount of unmodified protein materials, purification of protein isolate may be required before proceeding with the embodiment of the present invention. Specifically, an antecedent protein concentration or separation via hydrolysis may be required and can also be further purified by activated carbon or adsorbent resin. Preferably a starting protein isolate material is comprised of more than 50% (w/w) protein, more preferably 90% (w/w) protein. Protein isolates are used as the starting material, the embodiment of the present invention encompasses the isolation and concentration of low-molecular-weight oligopeptides for industrial applications from the starting material.

Dissolve protein isolate in 5 to 20 weight equivalents of alkali solution adjusted within pH 5 to 9 with a saturated alkaline solution at 20 to 45° C. Preferably, the alkaline solution comprises an alkaline material of sodium hydroxide, calcium hydroxide, magnesium hydroxide, potassium hydroxide or mixtures thereof. More preferably, the alkaline solution comprises sodium hydroxide.

Treat the solution by ultra-high temperature processing sterilization at 130 to 150° C. for 15 to 60 seconds. Cool the solution temperature to 40 to 70° C.

Maintain the solution temperature at 40 to 70° C. and the Brix at 5 to 20° Bx by adjusting with a non-reducing sugar. Examples of non-reducing sugars include, but are not limited to raffinose, stachyose, sucrose and verbascose. Add hydrolyzing agent or mixtures thereof at 1 to 15% (w/w) of the protein isolate. Stir the mixture for 0.5 to 8.0 hours. Hydrolyzing agent for use in the processes of the present invention may include enzymes, including proteases. The hydrolysis is preferably carried out by protease treatment Here protease animal origin, plant origin or microbial origin, can be appropriately selected based on the raw material protein source. Alternatively, a combination of enzymes originating from different source organisms may more efficiently result in hydrolysis to increase the percentage of resulting dipeptides and tripeptides. Suitable proteases may include: metalloendoproteases such as bacillolysin, Neutrase®, Maxazyme N P DS®; serine endoprotease such as trypsin, chymotrypsin, subtilisin (also subtilisin B, subtilopeptidase B, subtilopeptidase C, Nagarse, Nagarse proteinase, subtilisin Novo, bacterial proteinase Novo subtilisin A subtilopeptidase A alcalase Novo, similar enzymes are produced by various Bacillus subtilis strains and other Bacillus species and commercially available under names Alcalase® or Protex) Streptomyces alkaline protease, Bioplase®, Protease P®; cystein endopeptidase such as papain or bromelain; neutral proteases such as Streptomyces neutral protease, Aspergillus neutral protease, thermoase; acid proteases such as pepsin, Aspergillus acid protease, Sumichumu AP®, and; aminopeptidase such as Flavourzyme®, Sumizyme® FP, Corolase LAP® Peptidase 436PP436P and Peptidase 433PP433P (Biocatalysts, Wales, UK) or other aminopeptidases produced by other microorganisms than Aspergilli, for example Bacilli and Lactobacilli. Neutrase® is somewhat less preferred due to the presence of amylase side activities. Most preferably, the hydrolyzing agent is Alcalase®. Reaction pH, reaction temperature of the protease treatment may be altered to suit the characteristics of the protease used, but generally it is possible to carry out the reaction at a pH between 6 and 8 and temperature 40 to 70° C. The degree of hydrolysis is the extent to which peptide bonds are broken by the enzymatic hydrolysis reaction. The degree of hydrolysis most preferably between 10 and 50%.

Adjust the temperature of the reaction mixture to a temperature 75 to 95° C. and maintain for 10 to 30 minutes.

Cool the reaction mixture to room temperature and treat for 0.5 to 4.0 hours by cross-flow microfiltration using 300 to 3,000 Daltons molecular weight cut-off membrane with 0.2 to 1.0 mPa pulsating flow pressure. Maintain the Brix of the filtrate at 4 to 20° Bx.

Wash the filtrate and retentate with 1 to 15 equivalents of water and collect the filtrate.

Treat the filtrate by cross-flow nanofiltration using 150 to 300 Daltons molecular weight cut-off membrane with 0.3 to 0.8 mPa pulsating flow pressure at room temperature. Maintain the Brix of the retentate at 10 to 35° Bx

Collect the retentate.

Ultra-high temperature process sterilize the retentate at 130 to 150° C. for 15 to 60 seconds.

Spray dry the retentate. Preferably, with built-in fluidized bed tower. More preferably, maintain the inlet temperature at 140 to 180° C., bed temperature at 60 to 100° C., exhaust temperature at 90 to 110° C. and pressure at −20 to −80 Pa.

Drum dry the concentrate at 70 to 100° C. for 10 to 30 minutes.

Material proportions may be adjusted as scale demands. Likewise, system conditions may be adjusted. Brix may be adjusted by alternative soluble solids. Molecular weight cut-off may be adjusted to meet specific application requirements. The step described herein the best mode can be performed in the same manner for plant or marine protein isolates to produce the oligopeptide concentrated isolate according to the present invention described above.

### INDUSTRIAL APPLICABILITY

Proteins make up all of the body's organs and are required for proper function of organ systems. All proteins are made up of amino acids, but differences in amino acid composition and sequence differentiate how proteins function. The body uses amino acids to construct specific proteins for specific functions in the maintenance of organ health. However, the body has no de novo route for synthesis of many necessary amino acids, therefore these essential amino acids must be obtained from dietary protein. The body must consistently digest, absorb and metabolize adequate dietary proteins to supply organs with the specific proteins needed to function. Several other challenges exist. Complete dietary proteins often come from animal sources and are accompanied by cholesterol, which can be a rate-limiting factor for dietary consumption. As the body ages, digestive function tends to decline resulting in inefficient absorption of large dietary proteins. Another difficulty is that absorbed dietary proteins are not stored by the body to be metabolized later, so if all amino acids necessary to construct specific proteins are not present in optimal ratios during the metabolism, protein synthesis is inefficient.

However, challenges of obtaining adequate dietary protein can be overcome. Protein isolate supplements can effectively supply proteins to the body “just-in-time.” Cholesterol can be eliminated by isolating proteins from complete, vegetarian sources. Dietary protein can be more easily digested and absorbed by consuming smaller peptide subunits of proteins. Protein synthesis can be made more efficient by consuming dietary protein with optimized amino acid balance.

Previous methods have not produced plant protein isolates that overcome the aforementioned challenges. Furthermore, these methods have imposed limitations on suitability for industrial applications.

The current invention offers a solution for achieving plant peptide concentrates in high-yield with physical, chemical and biological attributes suitable for industrial applications.

The scope of the invention includes, but is not limited to applications in pharmaceutical, preventative health, dietary supplement, functional food and beverage, pediatric nutrition, food additive, animal feed, fertilizer, antioxidant, antimicrobial, cosmetic, surfactant, adhesive and bio-fuel formulations.

The following examples are to further illustrate the invention, but the present invention is not limited to these specific implementations.

### Examples

### Example 1: Establishment of Yield

Establishment of method for enriched plant or marine protein isolate by (a) dissolving the starting material protein isolate in various ratios of dry isolate 1:10 (w/v) alkali solution adjusted within pH 5 to 9 with a sodium hydroxide at 25° C.; (b) followed by ultra-high temperature processing treatment from 130 to 150° C.; (c) subsequent hydrolysis by 1% Alcalase® at 40 to 70° C. and 5 to 20° Bx by adjusting with sucrose, stirring the hydrolysis mixture for 4 to 6 hours; (d) cooling 75 to 95° C. and maintaining temperature for 20 minutes; (e) followed by cooling to 25° C.; (f) then separation for 2 hours by cross-flow microfiltration using 300 to 3,000 Daltons molecular weight cut-off membrane with 0.2 to 1.0 mPa pulsating flow pressure with Brix parameters 4 to 15° Bx; (g) washing filtrate and retentate with various ratios of water, 1 to 15 equivalents of the filtrate (v/v); (h) collecting the filtrate and treating by cross-flow nanofiltration using 150 to 300 Daltons molecular weight cut-off membrane with 0.3 to 0.8 mPa pulsating flow pressure at 10 to 35° Bx; (i) collection of the retentate followed by treatment at 130 to 150° C. for 15 to 60 seconds; (j) then spray drying the retentate with a built-in fluidized bed tower at an inlet temperature at 140 to 180° C., bed temperature at 60 to 100° C., exhaust temperature at 90 to 110° C. and pressure at −20 to −80 Pa; (k) last drum drying the concentrate at 70 to 100° C. for 10 to 30 minutes. Yield was measured as total yield of isolation over total starting raw material (w/w) and optical density (OD) measurements were taken at 660 nm to measure fluid clarity before drying. A lower absorbance score indicates greater clarity.

TABLE 1A
Relationship between product water quantity after microfiltration and
product yield
Protein isolate was dissolved in various ratios of water after microfiltration
(g) and processed similarly through steps (h) to (k). Yield was measured
as total yield of isolation over total starting raw material (w/w).

| Protein isolate:water quantity ratio (w:w) | Product yield |
|---|---|
| 1:1 | 25.54% |
| 1:2 | 28.35% |
| 1:12 | 65.97% |
| 1:15 | 70.31% |

TABLE IB
Relationship between microfiltration Brix content and product yield
Protein isolate was maintained at various Brix concentration after
microfiltration (g) and processed similarly through steps (h) to (k).
Yield was measured as total yield of isolation over total starting raw
material (w/w).

| Brix (° Bx) | Product yield |
|---|---|
| 4 | 23.86% |
| 5 | 30.79% |
| 7 | 41.23% |
| 15 | 64.27% |

TABLE 1C
Relationship between nanofiltration Brix concentration solution clarity
Protein isolate was maintained at various Brix concentration after
nanofiltration (h) and processed similarly through steps (i) to (k). Optical
density (OD) measurements were taken at 660 nm to measure fluid
clarity before drying.

| Brix (° Bx) | Clarity (OD 660 nm) |
|---|---|
| 10 | 90.3 |
| 12 | 91.5 |
| 30 | 90.7 |
| 35 | 89.9 |

TABLE ID
Yield of microfiltration membrane separation versus centrifugation
Protein isolate was processed similarly through steps (a) to (k), where
steps (f) to (g) were compared to conventional centrifugation separation
methods. Yield was measured as total yield of isolation over total starting
raw material (w/w).

| | Microfiltration separation | Centrifugation |
|---|---|---|
| Brix (° Bx) | yield | separation yield |
| 4 | 23.86% | 10.11% |
| 5 | 30.79% | 15.32% |
| 7 | 41.23% | 19.29% |
| 15 | 64.27% | 31.98% |

TABLE IE
Clarity nanofiltration membrane separation versus centrifugation
Protein isolate was processed similarly through steps (a) to (k), where
steps (f) to (g) were compared to conventional centrifugation separation
methods. Optical density (OD) measurements were taken at 660 nm to
measure fluid clarity before drying.

| | | Centrifugation |
|---|---|---|
| | Nanofiltration separation | separation clarity |
| Brix (° Bx) | clarity (OD 660 nm) | (OD 660 nm) |
| 10 | 90.3 | 81.2 |
| 20 | 90.4 | 80.3 |
| 30 | 90.7 | 78.5 |
| 40 | 86.9 | 70.7 |

### Example 2: Oligopeptide Enrichment

Soy protein isolate was chosen as the raw starting material wherein yielding protein content is comprised of about 95% oligopeptides. The isolated protein content molecular weight distribution was assessed on multiple batch preparations using the methods disclosed in this invention. A conventional isolation method by centrifugation and commercial soy peptide products were also assessed for comparison. Measurement of protein content, with respect to the dry weight of the various isolated protein material. The weight of the crude protein mass was measured by the Kjeldahl method, it expressed in weight percent. In addition, nitrogen coefficient it was 6.25. The molecular weight distribution of soy protein fraction of the hydrolyzate, it was measured by HPLC method using the following gel filtration column. The set an HPLC system using a gel filtration column for peptide, was charged with a known peptide comprising a molecular weight marker, to determine the calibration curve at the retention time of the relationship between the molecular weight. The isolate was diluted two-fold with gel filtration solvent (1% SDS in 10 mM phosphate buffer, pH 8.0) and 5 μL was applied it to the HPLC column (GE Healthcare Superdex Peptide 7.5 300GL). The column temperature was 25° C., flow rate 0.25 mL per minute and detection wavelength 220 nm. The percentage of molecular weight to the total amount of peptides and free amino acids in the isolate were calculated for the area of the entire absorbance in the time range.

TABLE 2
Isolate Molecular Weight Distribution

| | Molecular | Molecular | Molecular |
|---|---|---|---|
| | weight <150 | weight | weight |
| Preparation | Daltons | 150-1500 Daltons | >3000 Daltons |
| 1 | 97.58% | 1.97% | 0.46% |
| 2 | 95.50% | 1.91% | 0.62% |
| 3 | 94.47% | 2.46% | 0.57% |
| Conventional Method | 70.32% | 4.63% | 0.05% |
| Commercial Product | 87.79% | 11.90% | 0.30% |
| A | | | |
| Commercial Product | 86.79% | 13.21% | 0.00% |
| B | | | |

### Example 3: Uniform Granules

The isolated product granular particle size and moisture content were assessed on multiple batch preparations using the methods disclosed in this invention. Uniform granules reduce dust produced and clogs to spray drying tower while increasing control of water content.

TABLE 3A
Various temperatures of spray drying tower under fixed exhaust
inlet temperature

| Inlet temperature (° C.) | Particle size (μm) | Moisture content (%) |
|---|---|---|
| 140 | 150 | 4.8 |
| 150 | 140 | 4.5 |
| 160 | 150 | 4.5 |
| 170 | 140 | 4.3 |
| 180 | 140 | 4.3 |

TABLE 3B
Various bed temperatures under fixed spray drying inlet temperature and
exhaust temperature

| Bed temperature (° C.) | Particle size (μm) | Moisture content (%) |
|---|---|---|
| 60 | 80 | 5.5 |
| 70 | 60 | 6.0 |
| 80 | 60 | 5.8 |
| 90 | 60 | 6.3 |
| 100 | 60 | 5.2 |

TABLE 3C
Various drum drying temperatures under fixed inlet air temperature,
exhaust temperature and bed temperature

| Drum drying temperature | | |
|---|---|---|
| (° C.) | Particle size (μm) | Moisture content (%) |
| 70 | 50 | 4.5 |
| 75 | 60 | 4.5 |
| 80 | 60 | 4.3 |
| 90 | 60 | 3.8 |
| 100 | 60 | 3.5 |

TABLE 3D
Various drum drying times under fixed inlet air temperature, exhaust
temperature and bed temperature

| Drum drying time (min) | Particle size (μm) | Moisture content (%) |
|---|---|---|
| 10 | 50 | 4.0 |
| 15 | 60 | 3.8 |
| 20 | 60 | 3.6 |
| 25 | 60 | 3.0 |
| 30 | 60 | 3.0 |

### Example 4: Solubility

The isolated product was reconstituted in 1:10 in water (w/v). pH (adjusted to the appropriate level with diluted NaOH or HCl) and temperature were adjusted and solution clarity was assessed by optical density (OD) measurements at 610 nm on multiple batch preparations using the methods disclosed in this invention. A lower absorbance score indicates greater clarity. Solubility was assessed as the protein content of the dispersions, measured by nitrogen determination. 10 m L aliquots were transferred to pre-weighed centrifuge tubes and centrifuged at 7,800 g for 10 minutes to sediment the insoluble material. The protein content of the supernatant was measured by nitrogen content. The pellet material was dried overnight in an oven set at 100° C. and the weight of dry pellet material was recorded. Solubility (%) was calculated by (% protein in supernatant/% protein in initial dispersion)×100.

TABLE 4A
Product Solubility

| pH | Solubility (%) | OD (610 nm) |
|---|---|---|
| 2 | 23.7 | 1.353 |
| 4 | 93.9 | 0.031 |
| 6 | 100.0 | 0.019 |
| 8 | 90.7 | 0.039 |
| 10 | 29.6 | 1.237 |

While the invention has been explained in relation to its preferred embodiments, it is to be understood that various modifications thereof will become apparent to those skilled in the art upon reading the description. Therefore, it is to be understood that the invention disclosed herein is intended to cover such modifications as fall within the scope of the appended claims.

The present invention provides relates to plant and marine protein oligopeptide enriched isolates and processing for producing the same composed of high-yield method of isolating a granular, free-flowing, non-dusting, low-molecular-weight oligopeptides that is soluble, clear and heat-stable in an acidic aqeuous environment. Modifications are possible within the scope of this invention.

1. An enriched plant or marine oligopeptide isolate having a protein content of at least about 75 wt % molecular weight distribution of 150 to 1,500 Daltons, less than 5 wt %>3,000 Daltons and less than 5 wt % free amino acids of <150 Daltons, prepared by a method consisting of:
(a) Dissolve protein isolate starting material in 5 to 20 weight equivalents of alkali solution adjusted within pH 5 to 9 with a saturated alkaline solution at 20 to 45° C.;
(b) Treat the solution described in (a) by ultra-high temperature processing sterilization at 130 to 150° C. for 15 to 60 seconds then cool to 40 to 70° C.;
(c) Maintain solution described in (b) at 40 to 70° C. and 5 to 20° Bx and hydrolyzed with a hydrolyzing agent or mixtures thereof at 1 to 15% of the protein isolate (w/w), stirred for 0.5 to 8.0 hours;
(d) Adjust the temperature of the reaction mixture described in (c) to 75 to 95° C. and maintain for 10 to 30 minutes;
(e) Cool the reaction mixture described in (d) to room temperature and treat for 0.5 to 4.0 hours by cross-flow microfiltration using 300 to 3,000 Daltons molecular weight cut-off membrane with 0.2 to 1.0 m Pa pulsating flow pressure while maintaining the Brix of the filtrate at 4 to 20° Bx;
(f) Wash the filtrate and retentate from (e) with 1 to 15 equivalents of water and collect the filtrate;
(g) Treat the filtrate from (f) by cross-flow nanofiltration using 150 to 300 Daltons molecular weight cutoff membrane with 0.3 to 0.8 mPa pulsating flow pressure at room temperature, while maintaining Brix of the retentate at 10 to 35° Bx;
(h) Collect the retentate from (g);
(i) Ultra-high temperature process sterilize the retentate from (h) at 130 to 150° C. for 15 to 60 seconds;
(j) Spray dry the retentate from (i), and;
(k) Drum dry the concentrate from (j) at 70 to 100° C. for 10 to 30 minutes.

2. The enriched protein isolate as set forth in claim 1 wherein the isolate exhibits uniform granular particle size of from about 40 to 60 μm.

3. The enriched protein isolate as set forth in claim 2 wherein the granules exhibit a moisture content under 5%.

4. The enriched protein isolate as set forth in claim 3 wherein the isolate has greater than 90% solubility in an aqueous environment having a pH range from about pH 3.0 to 8.5.

5. An enriched plant or marine oligopeptide isolate having a protein content of at least about 85 wt % molecular weight distribution of 150 to 1,500 Daltons, less than 1 wt %>3,000 Daltons and less than 3 wt % free amino acids <150 Daltons, prepared by a method consisting of:
(a) Dissolve protein isolate starting material in 5 to 20 weight equivalents of alkali solution adjusted within pH 5 to 9 with a saturated alkaline solution at 20 to 45° C.;
(b) Treat the solution described in (a) by ultra-high temperature processing sterilization at 130 to 150° C. for 15 to 60 seconds then cool to 40 to 70° C.;
(c) Maintain solution described in (b) at 40 to 70° C. and 5 to 20° Bx and hydrolyzed with a hydrolyzing agent or mixtures thereof at 1 to 15% of the protein isolate (w/w), stirred for 0.5 to 8.0 hours;
(d) Adjust the temperature of the reaction mixture described in (c) to 75 to 95° C. and maintain for 10 to 30 minutes;
(e) Cool the reaction mixture described in (d) to room temperature and treat for 0.5 to 4.0 hours by cross-flow microfiltration using 300 to 3,000 Daltons molecular weight cut-off membrane with 0.2 to 1.0 m Pa pulsating flow pressure while maintaining the Brix of the filtrate at 4 to 20° Bx;
(f) Wash the filtrate and retentate from (e) with 1 to 15 equivalents of water and collect the filtrate;
(g) Treat the filtrate from (f) by cross-flow nanofiltration using 150 to 300 Daltons molecular weight cutoff membrane with 0.3 to 0.8 mPa pulsating flow pressure at room temperature, while maintaining Brix of the retentate at 10 to 35° Bx;
(h) Collect the retentate from (g);
(i) Ultra-high temperature process sterilize the retentate from (h) at 130 to 150° C. for 15 to 60 seconds;
(j) Spray dry the retentate from (i), and;
(k) Drum dry the concentrate from (j) at 70 to 100° C. for 10 to 30 minutes.

6. The enriched protein isolate as set forth in claim 5 wherein the isolate exhibits uniform granular particle size of from about 40 to 60 μm.

7. The enriched protein isolate as set forth in claim 6 wherein the granules exhibit a moisture content under 5%.

8. The enriched protein isolate as set forth in claim 7 wherein the isolate has greater than 90% solubility in an aqueous environment having a pH range from about pH 3.0 to 8.5.

9. An enriched plant or marine oligopeptide isolate having a protein content of at least about 90 wt % molecular weight distribution of 150 to 1,500 Daltons, less than 1 wt %>3,000 Daltons and less than 3 wt % free amino acids of <150 Daltons, prepared by a method consisting of:
(a) Dissolve protein isolate starting material in 5 to 20 weight equivalents of alkali solution adjusted within pH 5 to 9 with a saturated alkaline solution at 20 to 45° C.;
(b) Treat the solution described in (a) by ultra-high temperature processing sterilization at 130 to 150° C. for 15 to 60 seconds then cool to 40 to 70° C.;
(c) Maintain solution described in (b) at 40 to 70° C. and 5 to 20° Bx and hydrolyzed with a hydrolyzing agent or mixtures thereof at 1 to 15% of the protein isolate (w/w), stirred for 0.5 to 8.0 hours;
(d) Adjust the temperature of the reaction mixture described in (c) to 75 to 95° C. and maintain for 10 to 30 minutes;
(e) Cool the reaction mixture described in (d) to room temperature and treat for 0.5 to 4.0 hours by cross-flow microfiltration using 300 to 3,000 Daltons molecular weight cut-off membrane with 0.2 to 1.0 m Pa pulsating flow pressure while maintaining the Brix of the filtrate at 4 to 20° Bx;
(f) Wash the filtrate and retentate from (e) with 1 to 15 equivalents of water and collect the filtrate;
(g) Treat the filtrate from (f) by cross-flow nanofiltration using 150 to 300 Daltons molecular weight cutoff membrane with 0.3 to 0.8 mPa pulsating flow pressure at room temperature, while maintaining Brix of the retentate at 10 to 35° Bx;
(h) Collect the retentate from (g);
(i) Ultra-high temperature process sterilize the retentate from (h) at 130 to 150° C. for 15 to 60 seconds;
(j) Spray dry the retentate from (i), and;
(k) Drum dry the concentrate from (j) at 70 to 100° C. for 10 to 30 minutes.

10. The enriched protein isolate as set forth in claim 9 wherein the isolate exhibits uniform granular particle size of from about 40 to 60 μm.

11. The enriched protein isolate as set forth in claim 10 wherein the granules exhibit a moisture content under 5%.

12. The enriched protein isolate as set forth in claim 11 wherein the isolate has greater than 90% solubility in an aqueous environment having a pH range from about pH 3.0 to 8.5.

13. An enriched plant or marine oligopeptide isolate having a protein content of at least about 95 wt % molecular weight distribution of 150 to 1,500 Daltons, less than 1 wt %>3,000 Daltons and less than 3 wt % free amino acids of <150 Daltons, prepared by a method consisting of:
(a) Dissolve protein isolate starting material in 5 to 20 weight equivalents of alkali solution adjusted within pH 5 to 9 with a saturated alkaline solution at 20 to 45° C.;
(b) Treat the solution described in (a) by ultra-high temperature processing sterilization at 130 to 150° C. for 15 to 60 seconds then cool to 40 to 70° C.;
(c) Maintain solution described in (b) at 40 to 70° C. and 5 to 20° Bx and hydrolyzed with a hydrolyzing agent or mixtures thereof at 1 to 15% of the protein isolate (w/w), stirred for 0.5 to 8.0 hours;
(d) Adjust the temperature of the reaction mixture described in (c) to 75 to 95° C. and maintain for 10 to 30 minutes;
(e) Cool the reaction mixture described in (d) to room temperature and treat for 0.5 to 4.0 hours by cross-flow microfiltration using 300 to 3,000 Daltons molecular weight cut-off membrane with 0.2 to 1.0 m Pa pulsating flow pressure while maintaining the Brix of the filtrate at 4 to 20° Bx;
(f) Wash the filtrate and retentate from (e) with 1 to 15 equivalents of water and collect the filtrate;
(g) Treat the filtrate from (f) by cross-flow nanofiltration using 150 to 300 Daltons molecular weight cutoff membrane with 0.3 to 0.8 mPa pulsating flow pressure at room temperature, while maintaining Brix of the retentate at 10 to 35° Bx;
(h) Collect the retentate from (g);
(i) Ultra-high temperature process sterilize the retentate from (h) at 130 to 150° C. for 15 to 60 seconds;
(j) Spray dry the retentate from (i), and;
(k) Drum dry the concentrate from (j) at 70 to 100° C. for 10 to 30 minutes.

14. The enriched protein isolate as set forth in claim 13 wherein the isolate exhibits uniform granular particle size of from about 40 to 60 μm.

15. The enriched protein isolate as set forth in claim 14 wherein the granules exhibit a moisture content under 5%.

16. The enriched protein isolate as set forth in claim 15 wherein the isolate has greater than 90% solubility in an aqueous environment having a pH range from about pH 3.0 to 8.5.

17. An enriched plant or marine oligopeptide isolate having a protein content of at least about 97 wt % molecular weight distribution of 150 to 1,500 Daltons, less than 1 wt %>3,000 Daltons and <2.5 wt % free amino acids of <150 Daltons, prepared by a method consisting of:
(a) Dissolve protein isolate starting material in 5 to 20 weight equivalents of alkali solution adjusted within pH 5 to 9 with a saturated alkaline solution at 20 to 45° C.;
(b) Treat the solution described in (a) by ultra-high temperature processing sterilization at 130 to 150° C. for 15 to 60 seconds then cool to 40 to 70° C.;
(c) Maintain solution described in (b) at 40 to 70° C. and 5 to 20° Bx and hydrolyzed with a hydrolyzing agent or mixtures thereof at 1 to 15% of the protein isolate (w/w), stirred for 0.5 to 8.0 hours;
(d) Adjust the temperature of the reaction mixture described in (c) to 75 to 95° C. and maintain for 10 to 30 minutes;
(e) Cool the reaction mixture described in (d) to room temperature and treat for 0.5 to 4.0 hours by cross-flow microfiltration using 300 to 3,000 Daltons molecular weight cut-off membrane with 0.2 to 1.0 m Pa pulsating flow pressure while maintaining the Brix of the filtrate at 4 to 20° Bx;
(f) Wash the filtrate and retentate from (e) with 1 to 15 equivalents of water and collect the filtrate;
(g) Treat the filtrate from (f) by cross-flow nanofiltration using 150 to 300 Daltons molecular weight cutoff membrane with 0.3 to 0.8 mPa pulsating flow pressure at room temperature, while maintaining Brix of the retentate at 10 to 35° Bx;
(h) Collect the retentate from (g);
(i) Ultra-high temperature process sterilize the retentate from (h) at 130 to 150° C. for 15 to 60 seconds;
(j) Spray dry the retentate from (i), and;
(k) Drum dry the concentrate from (j) at 70 to 100° C. for 10 to 30 minutes.

18. The enriched protein isolate as setforth in claim 17 wherein the isolate exhibits uniform granular particle size of from about 40 to 60 μm.

19. The enriched protein isolate as set forth in claim 18 wherein the granules exhibit a moisture content under 5%.

20. The enriched protein isolate as set forth in claim 19 wherein the isolate has greater than 90% solubility in an aqueous environment having a pH range from about pH 3.0 to 8.5.
Material proportions may be adjusted as scale demands. Likewise, system conditions may be adjusted. Brix may be adjusted by alternative soluble solids. Molecular weight cut-off may be adjusted to meet specific application requirements. The step described herein the best mode can be performed in the same manner for plant or marine protein isolates to produce the oligopeptide concentrated isolate according to the present invention described above.
